SEQUENCE LISTING (9 sequences)

SEQ ID NO: 1 - protein, 15 aa, Unknown
  Marker peptides from the rmLT sequence
MPRGHNEYFDRGTQM

SEQ ID NO: 2 - protein, 15 aa, Unknown
  Marker peptides from the rmLT sequence
NINLYDHARGTQTGF

SEQ ID NO: 3 - protein, 15 aa, Unknown
  Marker peptides from the rmLT sequence
VRYDDGYVSTSLSLR

SEQ ID NO: 4 - protein, 15 aa, Unknown
  Marker peptides from the rmLT sequence
VSALGGIPYSQIYGW

SEQ ID NO: 5 - protein, 15 aa, Unknown
  Marker peptides from the rmLT sequence
GVIDERLHRNREYRD

SEQ ID NO: 6 - protein, 10 aa, Unknown
  Marker peptides from the rmLT sequence
GYRLAGFPPD

SEQ ID NO: 7 - protein, 10 aa, Unknown
  Marker peptides from the rmLT sequence
PPDHQAWREE

SEQ ID NO: 8 - protein, 17 aa, Unknown
  Marker peptides from the rmLT sequence
GYRLAGFPPDHQAWREE

SEQ ID NO: 9 - protein, 15 aa, Unknown
  Marker peptides from the rmLT sequence
YQSEVDIYNRIRNEL

(19) United States
(12) Patent Application Publication
(10) Pub. No.: US 2004/0170637 A1
(43) Pub. Date: Sep. 2, 2004

(54) Use of rmLT as a marker antigen for vaccines and as a synergistic adjuvant with AMPHIGEN

(76) Inventor: David Scott McVey, Beatrice, NE (US)

Correspondence Address: SCULLY SCOTT MURPHY & PRESSER, PC, 400 GARDEN CITY PLAZA, GARDEN CITY, NY, 11530

(21) Appl. No.: 10/714,679

(22) Filed: Nov. 14, 2003

Related U.S. Application Data
(60) Provisional application No. 60/426,421, filed on Nov. 14, 2002.

(51) Int. Cl. G01N 33/53; A61K 39/00; A61K 39/38

(52) U.S. Cl. 424/184.1

(57) ABSTRACT

The present invention provides methods which permit animal owners or food producers to readily establish the vaccination status of animals. Specifically, the present invention provides a method for determining whether an animal has been vaccinated with a vaccine which includes an immunogen and a substantially non-toxic rmLT by detecting the presence of antibodies or immune cells in an animal that are specific for the rmLT. Furthermore, the present invention provides a method for marking a vaccine composition by including in the vaccine composition a substantially non-toxic rmLT and detecting the presence of antibodies or immune cells in animals administered with such vaccine composition. Moreover, the present invention provides a method for enhancing the immunoprotective effects of an immunogen in a vaccine by including in the vaccine a substantially non-toxic rmLT. Vaccine compositions containing an immunogen, a substantially non-toxic rmLT and an oil-in-water emulsion adjuvant such as AMPHIGEN®, are also provided.

### CROSS REFERENCE TO RELATED APPLICATION

This application claims the benefit of U.S. Provisional Application Serial No. 60/426,421, filed on Nov. 14, 2002.

### FIELD OF THE INVENTION

This invention relates to the use of substantially non-toxic mutants of E coli heat labile enterotoxin as marker antigens for vaccines. The present invention further relates to the use of non-toxic mutants of E. coli heat labile enterotoxin and AMPHIGEN® as adjuvants in vaccines to potentiate the immunoprotective effects of immunogens. Vaccine compositions containing an immunogen, a substantially non-toxic rmLT and an adjuvant (such as AMPHIGEN®) are also provided by the present invention.

### BACKGROUND OF THE INVENTION

A variety of vaccines have been developed and sold in recent years to protect useful animals such as cattle and pigs against infections caused by microorganisms. However, the vaccination history of an animal is not always recorded in the documents accompanying the animal. Detection of the presence of antibodies in an animal specific for an immunogen is sometimes inconclusive in determining whether the animal has been vaccinated in compliance with product label directions, regulatory guidelines or mandates, and management specifications. Therefore, there exists a need to develop a method for determining the vaccination status of animals.

Enterotoxigenic E. coli strains cause severe diarrhea and intestinal disorders thorough the production of enterotoxins. These toxins are of two types, one of which is referred to as the heat-stable toxin (ST) because it survives treatment at 10° C. A second toxin is heat labile (LT) and is remarkably similar to the cholera toxin. LT is composed of a single A subunit and five identical B subunits. The A subunit is responsible for the biological and enzymatic activity of the toxin but is incapable of binding to its target receptors alone. The B subunit acts by binding to the intestinal epithelial cells thereby facilitating penetration of the A subunit. The A subunit penetrates the cell membrane and causes activation of adenylate cyclase by NAD-dependent ADP-ribosylation of a GTP-binding protein. The clinical effect of this action is to cause massive fluid secretion into the intestine.

A bacterial chromosomal form of LT has been identified and sequenced by Pickett C. L. et al (J. Bacteriol. 169, 5180-5187, (1987). Efforts have been made to develop detoxified LT toxins capable of inducing a protective immune response against enterotoxigenic bacteria. Harford et al. (Eur. J. Biochem. 183: 311,1989) describe the production of a toxoid which contained a Ser-61-Phe substitution and a Gly-79-Lys substitution in the A subunit of a LT pathogenic for pigs. Tsuji et al. (J. Biol. Chem. 265: 22520, 1990) describe a mutant LT which contained a single substitution Glu-112-Lys in the A subunit, which mutation affects the toxicity yet does not change the immunogenicity of the protein. U.S. Pat. No. 6,019,982 to Clements describes a substantially non-toxic mutant LT which contains the single substitution, Arg-192-Gly, in the A subunit. U.S. Pat. No. 6,033,673 to Clements describes a substantially non-toxic mutant LT which contains the substitution Arg-192-Gly and the substitution Leu-211-Ala in the A subunit. U.S. Pat. No. 6,149,919 to Domenighini et al. describes detoxified, immunogenic mutant LT proteins which contain substitutions at one or more of Arg-7, Asp-9, Arg-11, His-44, Arg-54, Ser-61, His-70, His-107, Glu-110, Glu-112, Ser-114, Trp-127, Arg-146 or Arg-192.

In addition to being an immunogen, LT has also been reported as possessing adjuvant activity. U.S. Pat. No. 5,182,109 to Tamura et al. describes that LT administered intranasally enhanced the antibody titer against a co-administered antigen. Clements et al. also demonstrated the adjuvant activity of LT when administered orally with unrelated antigens (Vaccine 6: 269-277, 1988; Abstract No. B91, 88th Ann. Meet. Am. Soc. Microbiol., 1988). U.S. Pat. No. 6,019,982 and U.S. Pat. No. 6,033,673 both describe mutant LT proteins which are substantially nontoxic but have retained the adjuvant activity of the wild type LT when orally administered with an unrelated antigen.

Prior to the present invention, there has been no report of the use of a substantially nontoxic mutant LT as a marker antigen in vaccines or as a synergistic adjuvant with oil-in-water emulsions such as AMPHIGEN®.

### SUMMARY OF THE INVENTION

In one embodiment, the present invention provides a method for identifying an animal that has been vaccinated with an immunogen, which method includes the steps of forming a vaccine composition which includes the immunogen and a recombinant, substantially non-toxic mutant of E. coli heat labile enterotoxin (rmLT); administering the vaccine composition to animal subjects; and detecting the presence of antibodies or immune cells in an animal that are specific for the rmLT as indicative of an earlier vaccination of the animal with the immunogen.

In another embodiment, the present invention provides a method for determining whether an animal has been vaccinated with a vaccine composition which includes an immunogen and a recombinant, substantially non-toxic rmLT, by detecting the presence of antibodies or immune cells in the animal that are specific for the rmLT as indicative of an earlier vaccination of the animal with the vaccine composition.

In still another embodiment, the present invention provides a method for marking or identifying a vaccine composition which contains an immunogen by including in the vaccine composition a substantially non-toxic rmLT as a marker antigen, and subsequently detecting the presence of antibodies or immune cells specific for the rmLT in animals vaccinated with the vaccine composition.

In a further embodiment, the present invention provides methods of enhancing the immunoprotective effects of an immunogen in a vaccine composition by including in the vaccine composition a substantially non-toxic rmLT as adjuvant. Preferably, the vaccine composition also includes an oil-in-water emulsion adjuvant such as AMPHIGEN®.

Vaccine compositions, which include an immunogen, a substantially non-toxic rmLT and an adjuvant such as AMPHIGEN®, are also provided by the present invention.

### BRIEF DESCRIPTION OF THE DRAWINGS

FIG. 1. Mycoplasma hyopneumoniae serology results. Treatment groups 4, 5 and 6 received vaccine with the rmLT marker. Treatments 1-3 also received M. hyopneumoniae vaccine.

FIG. 2. Western Blot analysis expressed as % responders of serum samples from Treatment Groups 6 and 7.

FIG. 3. Actual Western Blots for FIG. 2 of Treatment Groups 6 and 7.

FIG. 4. Western Blot analysis of murine sera.

FIG. 5A-5C. Reactivity of rmLT peptides with pre- and post-immunization sera or mAb in ELISA.

FIG. 6. Average rmLT S/P ratio for pigs vaccinated on Days 0 and 14 with different M. hyopneumoniae vaccines or placebo. An S/P ratio >0.364 was considered positive. Results are only shown for T01, T02, T3 and T06.

### DETAILED DESCRIPTION OF THE INVENTION

The present invention provides methods which permit animal owners or animal food producers to readily establish the vaccination status of animals and determine whether an animal has been vaccinated in compliance with product label directions, regulatory guidelines and mandates, and management specifications.

In one embodiment, the present invention provides a method for identifying an animal that has been vaccinated with an immunogen. Such method includes the steps of providing a vaccine composition which includes the immunogen and a recombinant, substantially non-toxic mutant of E. coli heat labile enterotoxin (rmLT); administering the vaccine composition to animal subjects; and detecting the presence of antibodies or immune cells in an animal that are specific for the rmLT as indicative of an earlier vaccination of the animal with the immunogen.

In another embodiment, the present invention provides a method for determining whether an animal has been vaccinated with a vaccine composition which includes an immunogen and a recombinant, substantially non-toxic rmLT. The determination is based on a detection of the presence of antibodies or immune cells in the animal that are specific for the rmLT as indicative of an earlier vaccination of the animal with the vaccine composition.

In still another embodiment, the present invention provides a method for marking or identifying a vaccine composition which contains an immunogen by including in the vaccine composition a substantially non-toxic rmLT as a marker antigen, and subsequently detecting the presence of antibodies or immune cells specific for the rmLT in animals vaccinated with the vaccine composition.

In a further embodiment, the present invention provides methods of enhancing the immunoprotective effects of an immunogen in a vaccine composition by including in the vaccine composition a substantially non-toxic rmLT as adjuvant. Preferably, the vaccine composition also includes an oil-in-water emulsion adjuvant such as AMPHIGEN®.

Vaccine compositions which include an immunogen, a substantially non-toxic rmLT and an adjuvant such as AMPHIGEN® are also provided by the present invention.

For clarity of disclosure, and not by way of limitation, the detailed description of the invention is divided into the following subsections which describe or illustrate certain features, embodiments or applications of the invention.

### Definitions and Abbreviations

The terms “animal” and “animal subject” as used herein refer to all non-human animals, including food animals such as cattle, sheep, pig and poultry.

The term “cattle” as used herein refers to bovine animals including but not limited to steer, bulls, cows, and calves.

The term “LT” as used herein refers to E. coli heat labile enterotoxin. LT is composed of a single A subunit and five identical B subunits. The B subunit acts by binding to the intestinal epithelial cells thereby facilitating penetration of the A subunit. The A subunit penetrates the cell membrane and causes activation of adenylate cyclase by NAD-dependent ADP-ribosylation of a GTP-binding protein. The A and B subunits are encoded by the LT gene, wherein the portion encoding the B subunit adjacent to and downstream from th portion encoding the A subunit.

The term “wild type LT” refers to a naturally occurring, toxic E. coli heat labile enterotoxin. A wild type LT made by an E. coli strain pathogenic to and isolated from one animal species may slightly differ in the amino acid sequence from another wild type LT made by an E. coli strain pathogenic to and isolated from another animal species. See, e.g., U.S. Pat. No. 6,149,919 to Domenighini et al.

The term “mLT” refers to a mutant form of a naturally occurring LT, which is generally generated by genetic engineering.

The term “rmLT” refers to a recombinantly produced mutant form of a naturally occurring LT.

The term “substantially non-toxic” as used in connection with a rmLT refers to a toxicity that is substantially lower than the wild type counterpart. By “substantially lower” is meant that the rmLT should have a toxicity of less than 2%, or preferably 1%, or even more preferably, 0.1% of the wild type LT. The toxicity can be measured by evaluation of the morphological changes induced in Y1 cells or other cell types and cell lines susceptible to wild type LT. Y1 cells are adrenal tumor epithelial cells which become markedly more rounded when treated with a solution containing LT (Yasamure et al., Cancer res. 26: 529-535, 1966). Alternatively, the toxicity can be assessed by evaluating fluid secretion induced by rmLT in experimental mice, as described in U.S. Pat. No. 6,019,982 to Clements.

By “marking or identifying a vaccine composition” is meant that the vaccine composition is made identifiable by having a substantially non-toxic rmLT as a component, and as a result, animals administered with the vaccine composition can be identified by detecting the presence of antibodies or immune cells in the serum that are specific for the rmLT in the animals.

The term “immune cells” as used herein refers to cells of the immune system of animals that participate in the initiation and direction of an immune response, and includes particularly T cells and B cells.

The term “specific for the rmLT” as used in connection with antibodies or immune cells is meant that the antibodies and immune cells recognize and react with the rmLT or portions or fragments of the rmLT, but not with an unrelated antigen or the immunogen in the vaccine compositions or portions of such immunogen or unrelated antigen.

The term “portion” and “fragment” as used in connection with a protein or polypeptide refers to a peptide sequence of at least 7 or 8 amino acids in length.

The term “immunogen” as used herein refers to any antigen or composition, unrelated to LT or rmLT, that is immunogenic in an animal.

The term “immunogenic” as used in connection with an immunogen or a composition is meant the capacity of the immunogen or the composition to provoke an immune response in an animal against the immunogen or the composition (or the components of the composition). The immune response can be a cellular immune response mediated primarily by cytotoxic T-cells, or a humoral immune response mediated primarily by helper T-cells, which in turn activates B-cells leading to antibody production.

By “enhancing the immunoprotective effects of an immunogen” is meant that the protective immune response induced by the immunogen is potentiated either in magnitude (e.g., an increased production of antibodies), in duration (e.g. total time in which detectable antibodies are present), or in kind (e.g., production of a new antibody isotype or subtype). The enhancement in the immune response induced by an immunogen can be readily determined by those skilled in the art.

### Preparation of rmLT

rmLTs suitable for use in the present methods and vaccine compositions include those that are substantially non-toxic, i.e., exhibit a substantially lower toxicity as compared to the wild type LT, but have retained the immunological properties (such as immunogenicity and adjuvant activity) of the wild type LT. The substantially lower toxicity should be sufficiently low for the rmLT to be used in a vaccine composition without causing significant side effects such as diarrhea. By “substantially lower” is meant that the rmLT should have a toxicity of less than 2%, or preferably 1%, or even more preferably, 0.1% of the wild type LT.

A mutant LT (mLT) can be generated by introducing one or more nucleotide changes in the wild type LT gene sequence, which result in one or more substitutions, deletions, frame-shift mutations, or truncations of amino acids in the A or the B subunit. Wild type LT sequences are known in the art. See, e.g., U.S. Pat. No. 6,149,919 to Domenighini et al. Methods for introducing nucleotide changes in a gene sequence, either by random mutagenesis or site-directed mutagenesis, are also well-known in the art and are generally described in Sambrook et al. (2001) Molecular Cloning: A Laboratory Manual, Third Edition Cold Spring Harbor Laboratory Press: Cold Spring Harbor, N.Y.

Preferably, one or more nucleotide changes are introduced into the sequence encoding the A subunit to generate a mutant A subunit. According to the present invention, preferred mutant forms of the A subunit includes one or more substitutions at the following amino acid positions: Arg-7, Asp-9, Arg-11, His-44, Arg-54, Ser-61, His-70, His-107, Glu-110, Glu-112, Ser-114, Trp-127, Arg-146 and Arg-192. The amino acid positions are numbered based on the amino acid sequence of the A subunit, LT1-1A, disclosed in U.S. Pat. No. 6,149,919 to Domenighini et al. An especially preferred mutant A subunit includes a substitution at Arg-192, e.g., Arg-192-Gly, as described in U.S. Pat. No. 6,019,982 to Clements. Another preferred mutant A subunit includes a substitution at Arg-192 as well as a substitution at another amino acid position, e.g., Leu-211. An example of such double mutant is R192G-L211A, as described in U.S. Pat. No. 6,033,673 to Clements.

The LT gene sequence containing a desired mutation can be placed in a vector capable of recombinant expression of the mutant LT in an appropriate host cell such as an E. coli strain. To achieve a high level expression, a strong promoter can be used in place of the native promoter of the LT gene. For example, U.S. Pat. No. 6,019,982 describes the recombinant plasmid, pDF82, which expresses wild type LT under control of the native LT promoter, and the recombinant plasmid, pBD95 (ATCC Deposit No. 69683), which expresses rmLT R192G under the lac promoter. Recombinant mutant proteins produced can then be purified using any well-known purification procedures.

The nontoxicity of a rmLT can be verified by, e.g., its lack of inducement of morphological changes in Y1 cells, or its lack of inducement of net fluid secretion in experimental mice, as described in U.S. Pat. No. 6,019,982 to Clements.

### Use of rmLT as a Marker Antigen

In one embodiment, the present invention provides a method for identifying an animal which has been vaccinated with an immunogen by providing a vaccine composition which includes both the immunogen and a substantially non-toxic rmLT; administering the vaccine composition to animal subjects; and detecting the presence of antibodies or immune cells that are specific for the rmLT in an animal as an indication that the animal has been vaccinated with the immunogen.

In another embodiment, the present invention provides a method for determining whether an animal has been vaccinated with a vaccine composition which contains an immunogen and a substantially non-toxic rmLT by detecting the presence of antibodies or immune cells that are specific for the rmLT in the animal as an indication that the animal has been vaccinated with the vaccine composition.

In still another embodiment, the present invention provides a method for marking or identifying a vaccine composition which contains an immunogen by including in the vaccine composition a substantially non-toxic rmLT as a marker antigen, and subsequently detecting the presence antibodies or immune cells that are specific for the rmLT in animals administered with the vaccine composition.

Immunogens which are appropriate for use in accordance with the present invention includes antigens prepared from pathogenic bacteria such as Mycoplasma hyopneumonia, Haemophilus somnus, Haemophilus parasuis, Bordetella bronchiseptica, Actinobacillus pleuropneumonie, Pasteurella multocida, Manheimia hemolytica, Mycoplasma bovis, Mycoplasma galanacieum, Mycobacterium bovis, Mycobacterium paratuberculosis, Clostridial spp., Streptococcus uberis, Streptococcus suis, Staphylococcus aureus, Erysipelothrix rhusopathiae, Campylobacter spp., Fusobacterium necrophorum, Escherichia coli, Salmonella enterica serovars, Leptospira spp.; pathog nic fungi such as Candida; protozoa such as Cryptosporidium parvum, Neospora canium, Toxoplasma gondii, Eimeria spp.; helminths such as Ostertagia, Cooperia, Haemonchus, Fasciola, either in the form of an inactivated whole or partial cell preparation, or in the form of antigenic molecules obtained by genetic engineering techniques or chemical synthesis. Additional immunogens include pathogenic viruses such as Bovine herpesviruses-1,3,6, Bovine viral diarrhea virus types 1 and 2, Bovine parainfluenza virus, Bovine respiratory syncytial virus, bovine leukosis virus, rinderpest virus, foot and mouth disease virus, rabies virus, swine fever virus, African swine fever virus, Porcine parvovirus, PRRS virus, Porcine circovirus, influenza virus, swine vesicular disease virus, Techen fever virus, Pseudorabies virus, either in the form of an inactivated whole or partial virus preparation, or in the form of antigenic molecules obtained by genetic engineering techniques or chemical synthesis.

A preferred immunogen is a Mycoplasma hyopneumoniae (or M. hyo) immunogen, i.e., an antigen prepared from M. hyo, capable of inducing an immune response in an animal against the antigen.

The M. hyo immunogen that may be used in the present invention can include, for example, an inactivated whole or partial M. hyo cell preparation, one or more M. hyo derived polypeptides, or immunogenic fragments of such polypeptides, or one or more M. hyo nucleic acids encoding for one or more M. hyo derived polypeptides, or immunogenic-fragments thereof, and which nucleic acids are capable of being expressed in vivo in an animal.

A preferred M. hyo immunogen for use in the methods of the present invention is a M. hyo bacterin.

M. hyo bacterins can be prepared from M. hyo isolates. Numerous M. hyo isolates are known to those skilled in the art and are available from, e.g., the American Type Culture Collection, 10801 University Boulevard, Manassas, Va. 20110-2209. These include for example: ATTC Nos. 25095, 25617, 25934, 27714 and 27715. M. hyo isolates can also be obtained directly from naturally or experimentally infected animal (e.g., porcine) lung lesions using known techniques. To obtain a M. hyo bacterin, cells of a M. hyo isolate can be inactivated using a variety of known methods, e.g., with binary ethyleneimine (BEI) as described in U.S. Pat. No. 5,565,205, or with, for example, formalin, heat, beta propriolactone (BPL), irradiation or glutaraldehyde.

M. hyo bacterins suitable for use as an immunogen in the method of the present invention can also be obtained through various commercial sources. Such sources include but are not limited to: RESPIFEND (Fort Dodge, American Home Products), HYORESP (Merial Ltd), M+PAC (Schering Plough), PROSYSTEM M (Intervet), INGLEVAC M (Boehringer), RESPISURE® (Pfizer Inc.), RESPISURE ONE™, STELLAMUNE™ Mycoplasma (Pfizer Inc.), and STELLAMUNE ONE™ Mycoplasma (Pfizer Inc.).

A preferred source of M. hyo bacterin for use in the method of the present invention is RESPISURE® (Pfizer Inc.), RESPISURE ONE™ (Pfizer Inc.), STELLAMUNE™ Mycoplasma (Pfizer Inc.), and STELLAMUNE ONE™ Mycoplasma (Pfizer Inc.).

A particularly preferred source of M. hyo bacterin for use in the method of the present invention is RESPISURE ONE™ (Pfizer Inc.), containing strain P-5722-3 (NL1 042), acquired from Purdue University, USA, which is inactivated, preferably, with BEI.

The M. hyo immunogen suitable for use in the present methods of the present invention also includes a polypeptide of M. hyo such as, but not limited to, P46, P65, P97, P102, P70, P50 and P44, or an immunogenic fragment of such a polypeptide. Preferably, the immunogenic fragment of a M. hyo polypeptide includes a contiguous portion of the polypeptide of at least 7 or 8, preferably, at least 25, and more preferably, at least 50 amino acids.

Alternatively, the M. hyo immunogen includes a nucleic acid molecule encoding a polypeptide of M. hyo such as, but not limited to, P46, P65, P97, P102, P70, P50 and P44, or an immunogenic fragment of such a polypeptide.

In accordance with the present invention, an immunogen and an rmLT marker antigen are combined with a veterinary-acceptable carrier to form a vaccine composition. The term “a veterinary-acceptable carrier” includes any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others. Stabilizers include albumin, among others.

Adjuvants suitable for use in accordance with the present invention include, but are not limited to, the RIBI adjuvant system (Ribi Inc.), alum, aluminum hydroxide gel, cholesterol, oil-in-water emulsions such as AMPHIGEN® (Hydronics, USA); water-in-oil emulsions such as, e.g., Freund's complete and incomplete adjuvants; Block co-polymer (CytRx, Atlanta Ga.); SAF-M (Chiron, Emeryville Calif.); saponins such as, e.g., Quil A, QS-21 (Cambridge Biotech Inc., Cambridge Mass.), GPI-0100 (Galenica Pharmaceuticals, Inc., Birmingham, Ala.) or other saponin fractions; monophosphoryl lipid A; Avridine lipid-amine adjuvant; cholera toxin; or muramyl dipeptide, among many others.

A preferred adjuvant for use in the vaccine compositions in accordance with the present invention is an oil-in-water emulsion adjuvant, particularly, AMPHIGEN® (Hydronics, USA).

The immunogen, the rmLT marker antigen and the veterinary-acceptable carrier can be combined in any convenient and practical manner to form a vaccine composition, e.g., by admixture, solution, suspension, emulsification, encapsulation, absorption and the like, and can be made in formulations such as tablets, capsules, powder, syrup, suspensions that are suitable for injections, implantations, inhalations, ingestions or the like. When appropriate, the pharmaceutical compositions of the present invention should be made steril by well-known procedures.

The amount of rmLT in the vaccine composition should be immunizing-effective and should generally be in the range of 1-500 μg per dose; preferably, in the range of 20-200 μg per dose; and more preferably, about 100 μg per dose.

The amount of the immunogen in the vaccine composition should be effective to induce a protective immune response in an animal and depends on the nature of the immunogen. Generally speaking, the amount of a M. hyo bacterin that is effective as an immunogen contains about 1×10⁶ to about 5×10¹⁰ color changing units (CCU) per dose, preferably, about 5×10⁸ to 5×10¹⁰ CCU/dose. The amount of a M. hyo polypeptide or an immunogenic fragment of such polypeptide effective as an immunogen can generally be in the range of about 0.01 μg to about 200 μg per dose. The amount of a nucleic acid molecule encoding a M. hyo polypeptide or an immunogenic fragment of such polypeptide effective as an immunogen can generally be in the range of about 0.01 μg to about 200 μg per dose.

In accordance with the present invention, the vaccine composition can be administered to an animal by known routes, including the oral, intranasal, mucosal topical, transdermal, and parenteral (e.g., intravenous, intraperitoneal, intradermal, subcutaneous or intramuscular). Administration can also be achieved using needle-free delivery devices. Administration can be achieved using a combination of routes, e.g., first administration using a parental route and subsequent administration using a mucosal route. A preferred route of administration is intramuscular administration.

To determine the presence of antibodies or immune cells in an animal specific for rmLT, a sample is taken from the animal. Samples which can be used for detecting specific antibodies include the blood, the serum fraction of the blood, the milk, bile and other body fluids of the animal. Alternatively, meat juice from a slaughtered animal can also be used. Samples which can be used for detecting specific immune cells, particularly T-lymphocytes, include the blood, the lymph nodes, the spleen or other lymphoid tissues throughout the body.

It should be noted that the A subunit antigen of LT or E. coli strains which produce LT do not reach an animal in normal livestock management as a vaccine or medicament nor food chain and against which no antibodies or immune cells are formed naturally in the animal. Accordingly, in a preferred embodiment, the detection step of the present methods involves detection of antibodies or immune cells specific for the A subunit of LT.

Detection of specific antibodies can be carried out by using any enzyme-immunological or immunochemical detection method, such as DIPSTICK, ELISA (enzyme linked immunosorbent assay, EIA (enzyme immunoassay), RIA (radioimmunoassay), Western Blot analysis, immunohistological staining and the like. Depending upon the assay used, the antigens or the antibodies can be labeled by an enzyme, a fluorophore or a radioisotope. See, e.g., Coligan et al. Current Protocols in Immunology, John Wiley & Sons Inc., New York, N.Y. (1994); and Frye et al., Oncogen 4: 1153-1157, 1987. Preferably, the detection is carried out using DIPSTICK, wherein labeled antigens are immobilized on a test stick which is dipped into a sample of body fluid from the animal, such as meat juice released naturally by the slaughtered animal.

Antigens which can be used in the detection of antibodies can be the A subunit or the B subunit of the wild type or mutant LT, or an antigenic fragment of the A subunit or the B subunit of the wild type or mutant LT. An antigenic fragment generally includes a contiguous fragment of the A or the B subunit of at least about 7 or 8 amino acids.

According to the present invention, preferred antigens for use in the detection of antibodies against the A subunit antigen of LT include peptide fragments of the A subunit of LT. More preferably, the antigen used in the detection has a sequence as set forth in any one of SEQ ID NOs: 1-9. A combination of two or more antigens can be employed in the detection.

Detection of specific immune cells, e.g., lymphocytes can be carried out by whole cell assays, cellular activation marker expression, gene activation marker expression, release of cytokines and hormones, binding assays (e.g., tetramers), delayed-type hypersensitivities (skin reaction tests), flow cytometric techniques for identification of antigen-specific immune cells.

### Use of rmLT as an Adjuvant

In another embodiment, the present invention provides a method of enhancing the immunoprotective effects of an immunogen in a vaccine composition by including in the vaccine composition a substantially non-toxic rmLT.

In accordance with the present invention, the immunoprotective effects of a variety of immunogens can be potentiated by rmLT, such immunogens including antigens prepared from pathogenic bacteria such as Mycoplasma hyopneumonia, Haemophilus somnus, Haemophilus parasuis, Bordetella bronchiseptica, Actinobacillus pleuropneumonie, Pasteurella multocida, Manheimia hemolytica, Mycoplasma bovis, Mycoplasma galanacieum, Mycobacterium bovis, Mycobacterium paratuberculosis, Clostridial spp., Streptococcus uberis, Streptococcus suis, Staphylococcus aureus, Erysipelothrix rhusopathiae, Campylobacter spp., Fusobacterium necrophorum, Escherichia coli, Salmonella enterica serovars, Leptospira spp.; pathogenic fungi such as Candida; protozoa such as Cryptosporidium parvum, Neospora canium, Toxoplasma gondii, Eimeria spp.; helminths such as Ostertagia, Cooperia, Haemonchus, Fasciola, either in the form of an inactivated whole or partial cell preparation, or in the form of antigenic molecules obtained by genetic engineering techniques or chemical synthesis. Additional immunogens include pathogenic viruses such as Bovine herpesviruses-1,3,6, Bovine viral diarrhea virus types 1 and 2, Bovine parainfluenza virus, Bovine respiratory syncytial virus, bovine leukosis virus, rinderpest virus, foot and mouth disease virus, rabies virus, swine fever virus, African swine fever virus, Porcine parvovirus, PRRS virus, Porcine circovirus, influenza virus, swine vesicular disease virus, Techen fever virus, Pseudorabies virus, either in the form of an inactivated whole or partial virus preparation, or in the form of antigenic molecules obtained by genetic engineering techniques or chemical synthesis.

In a preferred embodiment, a substantially non-toxic rmLT is used to potentiate the immunoprotective effects of a M. hyo immunogen, i.e., an antigen prepared from M. hyo capable of inducing an immune response in an animal against the antigen. Such M. hyo immunogen can include, for example, an inactivated whole or partial M. hyo cell preparation, one or more M. hyo derived polypeptides, or immunogenic fragments of such polypeptides, or one or more M. hyo nucleic acids encoding for one or more M. hyo derived polypeptides, or immunogenic-fragments thereof, and which nucleic acids are capable of being expressed in vivo in an animal.

In a more preferred embodiment, the M. hyo immunogen is a M. hyo bacterin which can be obtained as described hereinabove and include those that are commercially available, e.g., RESPIFEND (Fort Dodge, American Home Products), HYORESP (Merial Ltd), M+PAC (Schering Plough), PROSYSTEM M (Intervet), INGLEVAC M (Boehringer), RESPISURE® (Pfizer Inc.), RESPISURE ONE™, STELLAMUNE Mycoplasma (Pfizer Inc.), and STELLAMUNE ONE™ Mycoplasma (Pfizer Inc.).

A preferred source of M. hyo bacterin for use in the method of the present invention is RESPISURE® (Pfizer Inc.), RESPISURE ONE™ (Pfizer Inc.), STELLAMUNE™ Mycoplasma (Pfizer Inc.), and STELLAMUNE ONE™ Mycoplasma (Pfizer Inc.). A particularly preferred source of M. hyo bacterin is RESPISURE ONE™ (Pfizer Inc.).

In accordance with the present invention, an immunogen and an rmLT are combined with a veterinary-acceptable carrier to form a vaccine composition. Suitable veterinary-acceptable carriers have been described hereinabove and include any and all solvents, dispersion media, coatings, adjuvants, stabilizing agents, diluents, preservatives, antibacterial and antifungal agents, isotonic agents, adsorption delaying agents, and the like.

Adjuvants suitable for use in the vaccine compositions in accordance with the present invention have also been described hereinabove. A preferred adjuvant is the oil-in-water emulsion adjuvant, AMPHIGEN® (Hydronics, USA).

The immunogen, the rmLT antigen and the veterinary-acceptable carrier can be combined in any convenient and practical manner to form a vaccine composition, by admixture, solution, suspension, emulsification, encapsulation, absorption and the like, and can be made in formulations such as tablets, capsules, powder, syrup, suspensions that are suitable for injections, implantations, inhalations, ingestions or the like.

The amount of rmLT in the vaccine composition should generally be in th range of 1-500 μg per dose; preferably, in the range of 20-200 μg per dose; and more preferably, about 100 μg per dose.

The amount of the immunogen in the vaccine composition should be effective to induce a protective immune response in an animal and depends on the nature of the immunogen. Generally speaking, the amount of a M. hyo bacterin that is effective as an immunogen contains about 1×10⁶ to about 5×10¹⁰ color changing units (CCU) per dose, preferably, about 5×10⁸ to 5×10¹⁰ CCU/dose. The amount of a M. hyo polypeptide or an immunogenic fragment of such polypeptide effective as an immunogen can generally be in the range of about 0.01 μg to about 200 μg per dose. The amount of a nucleic acid molecule encoding a M. hyo polypeptide or an immunogenic fragment of such polypeptide effective as an immunogen can generally be in the range of about 0.01 μg to about 200 μg per dose.

In accordance with the present invention, the vaccine composition can be administered to an animal by known routes, as described herein above. A preferred route of administration is intramuscular administration.

Vaccine compositions which include an immunogen, a substantially non-toxic mutant of E. coli heat labile enterotoxin and an oil-in-water emulsion adjuvant such as AMPHIGEN® (Hydronics, USA), are also provided by the present invention.

The present invention is further illustrated by the following non-limiting examples.

### EXAMPLE 1

### Preparation of a M. hyo Bacterin

The Mycoplasma hyopneumoniae production culture medium was prepared according to the following formula and procedure:

| | |
|---|---|
| ppLo broth without crystal violet | 81.00% ± 2% |
| Cysteine hydrochloride | 0.01% ± 0.005% |
| Yeast extract | 6.25% ± 0.2% |
| Dextrose (Technical or analytical grade) | 1.00% ± 0.2% |
| Irradiated, heat treated swine serum | 10.00% ± 1% |

The pH of the ppLo broth was adjusted to 7.0±0.4 and filter sterilized using a 0.2 micron filter. The yeast extract and ppLo broth were heat sterilized at a minimum of 121° C. for 15 to 60 minutes, and allowed to cool to below 60° C. The other components listed above were added as sterile additions.

An antigen production culture was initiated by thawing a frozen vial of stock Mycoplasma hyopneumoniae bacteria into the production medium. All cultures were incubated at 37±2° C. The initial culture expansions were performed in flasks (up to one liter) and then subsequently in fermentation vessels with pH regulated to approximately 7.0 and dissolved oxygen at 25% of maximum. The culture was expanded to approximately 1800 liters. Prior to inactivation, the culture was examined under a phase contrast microscope for characteristic morphology and Gram-stained preparations were examined for the presence of contamination.

The culture was free from contamination and yielded an antigen concentration of approximately 5×10⁹ CCU/mL as determined by a color changing units titration. At the end of the growth period, the culture was inactivated in the fermenter or holding vessel. The pH of the culture was raised to 7.8±0.2, and the pH was maintained within this range for one hour. At this time, a filter sterilized aqueous solution of 2-BromoEthylAminehydrobromide (BEA) was added to a final concentration of approximately 4.0 mM. In the presence of the elevated pH, the BEA is chemically changed to using BinaryEthylenelmine (BEI). The culture was incubated at 37±2° C. with constant agitation for 24 hours. After the 24-hour incubation, a filter sterilized aqueous solution of sodium thiosulfate was added to a final concentration of approximately 4 mM to neutralize excess BEI. The culture was incubated at 37±2° C. with constant agitation for an additional 24 hours.

The inactivated culture was transferred into sterile storage vessels and stored at 2-8° C. until assembled.

### EXAMPLE 2

### Preparation of an rmLT Antigen

Plasmid pUK21 was generated as follows for the recombinant production of rmLT R192G. Plasmid pCS95 contained the native regulatory elements of the LT gene and constitutively expressed the protein rmLT R192G in an E. coli host. This plasmid was constructed in a pUC-based vector, which carried the ampicillin resistant marker. The rmLT gene, including the native regulatory sequences, was excised from the pCS95 plasmid and cloned into another pUC based kanamycin resistant vector, pUK21, to yield the construct pUK21/rmLT. Plasmid pUK21/rmLT was deposited with the American Type Culture Collection (ATCC), 10801 University Blvd., Manassas, Va. 20110-2209, on Sep. 18, 2003, and has been assigned the accession number, ATCC # PTA-5546.

Plasmid pUK21/rmLT was transformed into E. coli host strain JM83 (genotype F^{- φ80}dlacZΔM15 Δ(lac-proAB) ara rpsL). Two tryptose soy agar plates were streaked for confluent growth of transformants (JM83/pUK21/rmLT) and incubated at 37° C. or 18 hours. One plate was harvested in 10 ml of Evans Medium and this harvest was used to inoculate a sterile 10-liter fermenter vessel. The Evans Medium contained (on a per liter basis):

Casamino Acids—20 gm

Yeast Extract—6 gm

NaCl—2.5 gm

K₂HPO₄—8.7 gm

Trace Mineral Salt Solution—1 ml

50% glucose—10 ml

The trace mineral salt solution contained 5% MgSO₄.7H₂O, 0.5% MnCl₂.4H₂O and 0.5% FeCL₃.6H₂O (all weight/volume). The medium also contained 100 μg of kanamycin sulfate per liter. The fermentation culture was maintained at 37° C. for 18 hours with moderate aeration. The cell mass was removed by centrifugation using a Wesffalia centrifuge. Prior to purification by affinity chromatography, the centrate was clarified by 0.2 μm depth filtration. This crude rmLT fraction was purified and effectively concentrated by affinity chromatography on a α-D-galactose resin. The TEAN elution buffer (pH=7.5) was:

0.05 M Tris

0.001M EDTA

0.003 M sodium azide

0.2 M NaCl

The eluted rmLT was subjected to diafiltration to remove the azide with a 10K A/G Tech hollow fiber filter against TEN buffer (pH=7.35):

0.05 M Tris

0.001M EDTA

0.2 M NaCl

The diafiltrate was filtered through a 0.2 μm filter. Lactose was added to 5% (weight/volume) and the preparation was lyophilized. The lyophilized material was stored at ambient room temperature until formulated in to the vaccines.

### EXAMPLE 3

The oil phase of the adjuvant was comprised of AMPHIGEN® Base (40% Soya Lecithin, 60% Mineral Oil), Drakeol (100% Mineral Oil), and two surfactants Span 80 and Tween 80. The AMPHIGEN® Base was blended with the Drakeol at 25% AMPHIGEN® to 75% Drakeol by volume. By volume, these two oils comprised 5±0.5% of the final volume of the vaccine. Span 80 was added at 1.2±0.3% of the final serial volume. Tween 80 is added at 2.8±0.3% of the final serial volume. The oil phase was allowed to mix 2 hours 37±2° C. prior to addition to the aqueous phase of the product (via emulsification). The aqueous phase of the vaccine included the bulk M. hyopneumoniae antigen prepared in Example 1. The bulk M. hyopneumoniae antigen was added to PBS. Before addition to the oil phase, sufficient rmLT protein was added to the aqueous phase to allow a final concentration of 10, 20 or 50 μg per dose of vaccine. The formulation of vaccines were:

PBS with/without rmLT—83.75%

Bulk M. hyopneumoniae antigen—11.25%

Oil adjuvant—5.00%

The aqueous components were added to the warm oil phase and emulsified by high-speed mixing.

### EXAMPLE 4

The detection of serum antibodies to the A subunit of the rmLT complex was accomplished by Western Immunoblot procedures. This was facilitated by use of monoclonal antibodies specific to the A and B subunits. The subunits were separated by polyacrylamide gel electrophoretic chromatography. 30 μg rmLT was loaded onto a 10% polyacrylamide bis-tris SDS gel along with appropriate molecular weight, monomeric standards. The gel underwent electrophoreses for 45 minutes at 150 V. The transfer of the rmLT protein from the gel to a nitrocellulose membrane was achieved by using an electrochemical gradient. The membrane was rinsed and blocked for 2 hours with 5% non-fat dry milk in TBS, followed by another rinse. Serum samples to be tested were diluted 1:800 in 5% non-fat dry milk/TBS, and were subsequently loaded onto the membrane using the Biorad@ multi-screen apparatus and allowed to incubate overnight with gentle agitation. Samples identified as 311 and 30 were used as positive and negative controls, respectively. The membrane was then rinsed and anti-swine-AP conjugate diluted 1:2000 in 5% non-fat dry milk/TBS. After a two-hour incubation, the membrane was rinsed and developed with chromophore to allow visualization of proteins recognized by antibodies. In this case, if antibodies to the A subunit were present in a serum sample, the band would be observed as stained. If the A subunit band was visible after appropriate development, the serum sample was considered positive.

### EXAMPLE 5

Detection of antibodies to M. hyopneumoniae antigens is achieved by use of a conventional, indirect ELISA test. Antigens were extracted from washed M. hyopneumonia cells by detergent (Tween 20 in phosphate-buffered saline). The extracted antigens are stored at −70° C. until used. The antigens were added to wells of a 96-well plate (such as an Immunol 2, BioTek). The results of the test were recorded as a ratio of the signal derived from an unknown, test sample to a positive control sample signal. Test negative and dilutent wells were also used as negative controls for the assay.

### EXAMPLE 6

Animals Fifty, apparently healthy, crossbred pigs (11 groups of 10 pigs each) without a history of disease caused by M. hyopneumoniae or vaccination against the same organism were obtained from Crystal Lean Farms, Villard, Minn. at approximately 12 to 16 days of age. Prior to the initiation of the study, the animals were double ear-tagged with an identical sequential number. Pigs were allocated to treatments and pens according to a random treatment allocation plan provided by Pfizer Biometrics and Data Management. Pigs were randomized to treatment groups following a randomized complete block design, using body weight as a blocking factor, and will be housed in pens by block.

Upon arrival at Terre Haute, pigs were treated according to label directions with ceftiofur sodium (Naxcel™) intramuscularly in the ham muscle for three consecutive days in order to prevent stress-related infections such as Streptococcus suis and to decrease the incidence of Haemophilus parasuis. Pigs were acclimatized for a minimum of five days prior to the initiation of the study. Pigs were fed a concentrated medicated diet, free of any known contaminants or pesticides and had free access to water. Approximately two weeks prior to the challenge, pigs were switched to a concentrated non-medicated diet.

Vaccination

The experimental vaccines used in this study are shown in the Table 1 below.

TABLE 1

| | | | | Marker |
|---|---|---|---|---|
| Group | Serial | Antigen | Marker* | concentration** |
| T04 | TBD | M. hyopneumoniae | rmLT | 10 μg per dose |
| T05 | TBD | M. hyopneumoniae | rmLT | 20 μg per dose |
| T06 | TBD | M. hyopneumoniae | rmLT | 50 μg per dose |
| T07 | TBD | M. hyopneumoniae | None | None |
| T08 | TBD | None | None | None |

*Equivalent of a licensed product, RESPISURE ®
**Three concentrations of each marker will be evaluated. Exact concentrations will be documented.

Animals were vaccinated twice with 2 ml of the appropriate vaccine or placebo by the intramuscular route (right neck muscle) on day 0 when pigs were approximately 3 weeks of age and on day 14 (left neck muscle) when pigs were approximately 5 weeks of age.

Blood Sampling

On Days −1 or 0, 13 or 14, 28, 42, 62 or 63 and 91, a blood sample (approximately 10 mL in serum separator tubes) was collected via venopuncture of the proximal vena cava/jugular vein from all pigs. On Day 91, the blood sample was collected from axillary vessels severed following deep anesthesia. Serum from each blood sample was stored at −20° C. until evaluated for antibody activity to M. hyopneumoniae and rmLT antigens.

Challenge

Pigs in treatment groups 06, 07 and 08 were challenged as described below.

Approximately 7 weeks following the second vaccination (Day 63), pigs were challenged via the intranasal route. On 3 consecutive days, pigs were challenged intranasally with 5 mL of the M. hyopneumoniae inoculum, 2.5 mL per nostril. Prior to challenge, pigs were anesthetized by an intramuscular injection of 4.4 mg per kg xylazine and 4.4 mg per kg tiletamine and zolazepam (Telazol®, Fort Dodge Laboratories Inc.; Fort Dodge, Iowa).

The challenge inoculum was a suspension of M. hyopneumoniae in a lung homogenate, (containing a derivative of M. hyopneumoniae strain 11). The inoculum was diluted in sterile Friis mycoplasmal media to achieve a 1:50 dilution, which contained approximately 10⁵ color changing units (CCU) per mL. Following challenge inoculation, all pigs were observed daily and any abnormalities were recorded.

Necropsy

Approximately 4 weeks after challenge (Day 91), all remaining animals were euthanized with a lethal, intravenous injection of pentobarbital sodium (3-10 ml) and were exsanguinated by cutting both axillary arteries. All major organs, excluding central nervous system, were examined grossly by a veterinarian or qualified personnel and all necropsy findings were recorded. Lungs were removed and evaluated grossly for characteristic lesions attributable to a M. hyopneumoniae infection. Lesions were sketched on a standard lung diagram.

Serology

Serology titer data were classified according to the positive or negative detection of antibodies to the rmLT marker at each time-point. The incidence of Positive/Negative status of the marker by time-point was summarized by treatment. The serological response to the M. hyopneumoniae component of the experimental vaccines was determined by ELISA using a Tween-20-extracted antigen preparation as the solid phase of the test. Response indices were generated, which indicate antibody responses relative to known positive and negative control sera.

Western Blot Analysis

Recombinant rmLT was subjected to SDS-PAGE and electrophoretically transferred to a nylon membrane. The blots were probed with antisera from these studies as well as monoclonal antibodies to provide evidence of both A and B subunit-specific antibody activity.

Percentage of Total Lung with Lesions

Percent gross involvement per each lung lobe were summarized and then weighted using the following ratios of individual lung lobes to total lung mass: left cranial 10%, left middle 10%, left caudal 25%, right cranial 10%, right middle 10%, right caudal 25%, and accessory 10%. The weighted lung lobe values were then summed across lobes to yield the Percentage of Total Lung with Lesions. The arcsin square root transformation was applied to the percentage of total lung with lesions prior to analysis. Percentage of Total Lung with Lesions was analyzed using a mixed linear model that includes the effects of block and treatment. Linear combinations of the parameter estimates (obtained from the model) were used in a priori contrasts after testing for a significant (P≦0.05, one-tailed) difference between Placebo (T08) and the average of the vaccinates (T04-T07). Comparisons were made between treatment groups. The 5% level of significance (P≦0.05, one-tailed) was used to assess statistical differences. Back transformed mean percentage of total lung with lesions was calculated from least squares means of the arcsin square root (% total lung with lesions).

| Schedule of Events | |
|---|---|
| Day 0 | 1^{st} Vaccination/Blood sample/T04-08 |
| Day 13 | 2^{nd} Vaccination/Blood sample |
| Day 28 | Blood sample |
| Day 43 | Blood sample |
| Day 63 | T06, T07, T08 Challenge/Blood sample |
| Day 91 | Necropsy/Blood sample |

Results

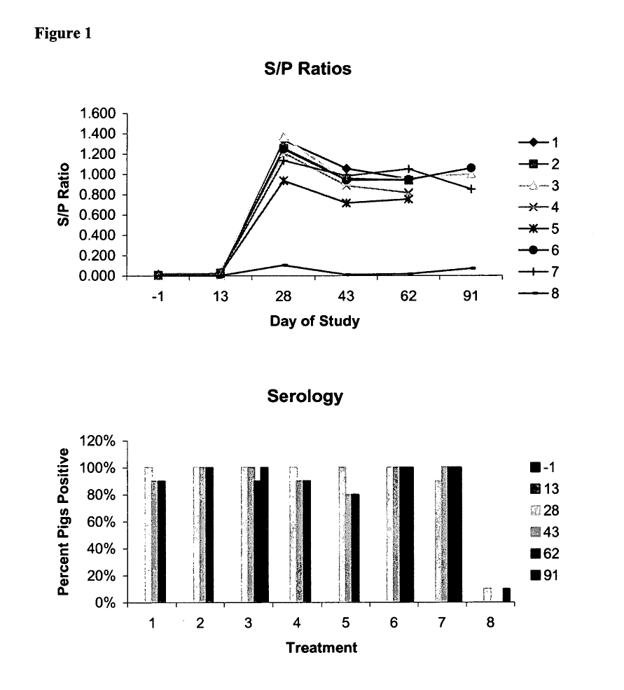

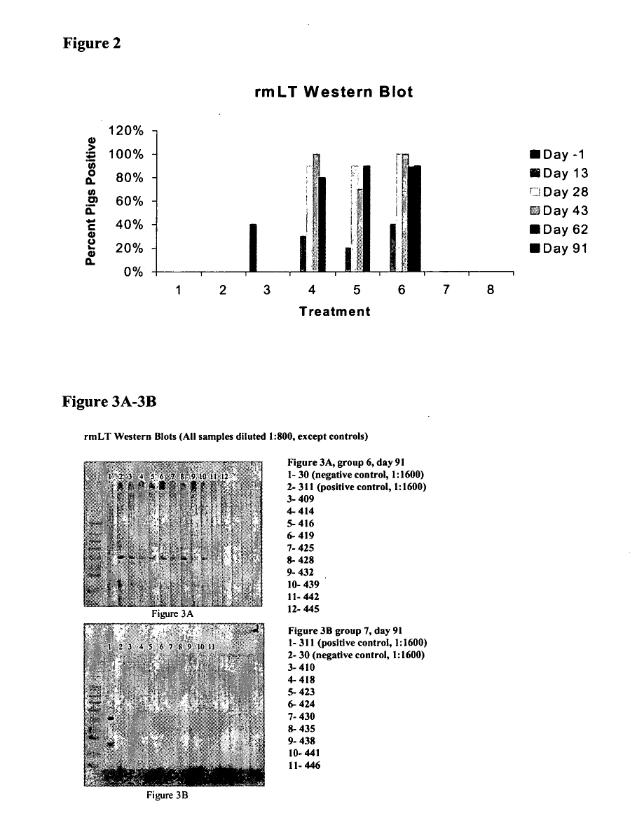

The serology results are depicted in FIG. 1 and FIG. 2. The Western immunoblots of treatment groups 06 and 07 are presented in FIG. 3. These data indicate that the rmLT protein is antigenic in the RESPISURE® formulations and consistently stimulates antibodies to the A subunit of the protein. In addition, antibody response to the mycoplasmal antigen is not compromised.

Macroscopic lung lesion scores were summarized in Table 2. These results clearly indicate that the combination of the rmLT marker protein and the M. hyopneumoniae antigen, together with the AMPHIGEN® adjuvant, resulted in a synergistic and profound protection.

TABLE 2

| | | Total Lung with Lesions | | |
|---|---|---|---|---|
| Treatment | N | LS Mean | SE | Range |
| 6 | 10 | 0.3 | 0.25 | 0-3 |
| 7 | 9 | 4.0 | 2.62 | 0-29.5 |
| 8 | 10 | 1.7 | 1.31 | 0-19.5 |

### EXAMPLE 7

Vaccination

Mice (12-week old CF-1 females) were vaccinated twice with 0.2 ml of the experimental vaccines subcutaneously on day 0 and on day 14. Serum was collected on day 21. The experimental vaccines used in this study are shown in the Table 3 below.

TABLE 3

| | | | | Marker |
|---|---|---|---|---|
| Group | Serial | Antigen | Marker* | concentration** |
| G2 | TBD | M. hyopneumoniae | rmLT | 10 μg per dose |
| G3 | TBD | M. hyopneumoniae | rmLT | 20 μg per dose |
| G16 | TBD | M. hyopneumoniae | rmLT | 50 μg per dose |
| G4, G5 | TBD | M. hyopneumoniae | None | None |
| G13 | TBD | None | None | None |

*Equivalent of a licensed product, RESPISURE ®
**Three concentrations of each marker will be evaluated. Exact concentrations will be documented.

Serology

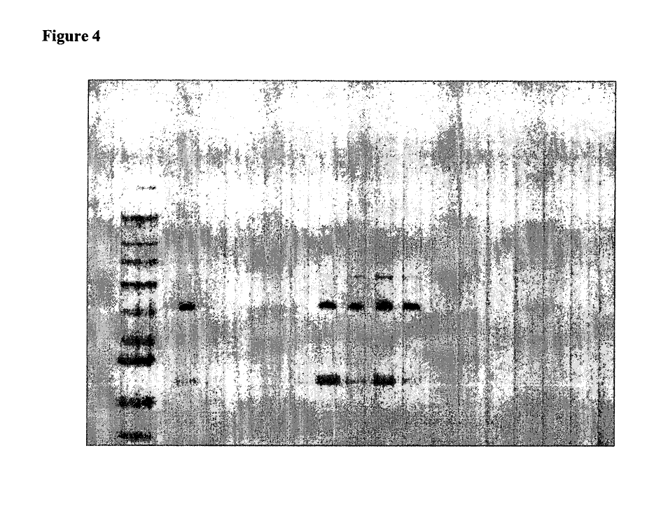

Serology titer data were classified according to the positive or negative detection of antibody to the rmLT marker in the serum collected on day 21. As can be seen from FIG. 4, mice also respond to the rmLT in the RESPISURE® vaccine formulation by producing antibody to the A subunit of the rmLT.

### EXAMPLE 8

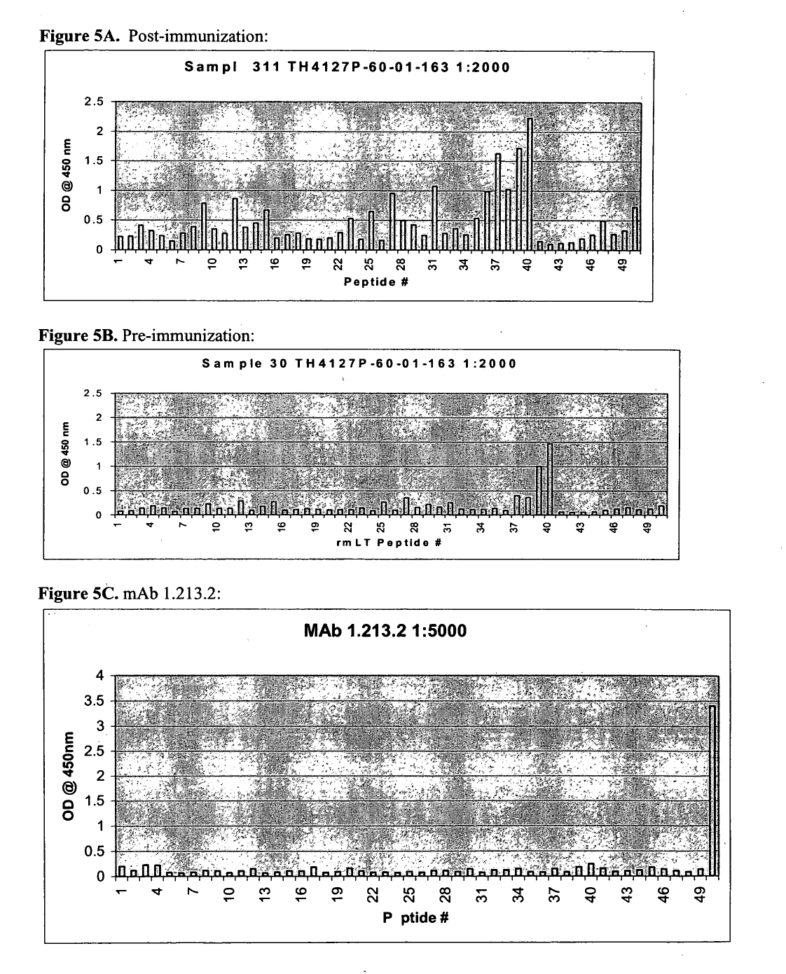

To identify the epitopes of the rmLT A subunit, we constructed a set of peptides (15-mers) that scanned the entire rmLT a subunit sequence, overlapping by five amino acids per peptide. For example, peptide 1 was a contiguous sequence of amino acids number 1 to 15; and peptide 2 was a contiguous sequence of amino acids number 10 to 25. This pattern was followed to cover the entire primary sequence of rmLT. The peptides were attached covalently to resin pins. The non-cleavable pins were held on a support frame to facilitate submersion of the pins in to 96-well plates. Thus each peptide on a pin became the solid phase of an ELISA assay to assess binding of the mAbs tested. The pin sets were tested against sera from pigs pre- and post-immunization and select mAbs. The results were presented in FIG. 5A-5C.

As shown in FIGS. 5A-5B, peptides 9, 12, 15, 27, 31 and 37 and 50 represent the epitopes that can be used as marker peptides from the rmLT sequence. The sequences of these peptides are:

| | |
|---|---|
| 9- MPRGHNEYFDRGTQM | (SEQ ID NO: 1) |
| 12- NINLYDHARGTQTGF | (SEQ ID NO: 2) |
| 15- VRYDDGYVSTSLSLR | (SEQ ID NO: 3) |
| 27- VSALGGIPYSQIYGW | (SEQ ID NO: 4) |
| 31- GVIDERLHRNREYRD | (SEQ ID NO: 5) |
| 36- GYRLAGFPPD | (SEQ ID NO: 6) |
| 37- PPDHQAWREE | (SEQ ID NO: 7) |
| 36/37 hybrid- GYRLAGFPPDHQAWREE | (SEQ ID NO: 8) |
| 50- YQSEVDIYNRIRNEL | (SEQ ID NO: 9) |

### EXAMPLE 9

### Results from 173 Study

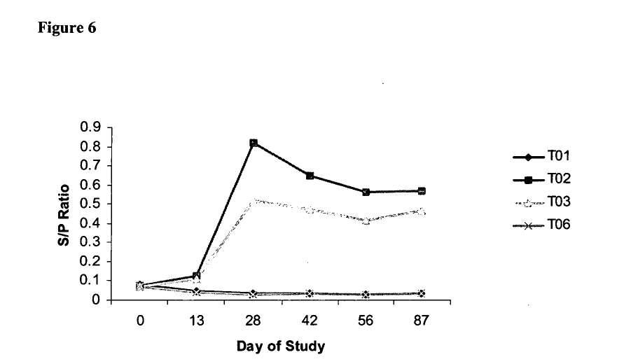

This a repeat of the first porcine study exemplified above that confirmed the positive serological response to the A subunit. The data in FIG. 6 and Table 4 demonstrate that the rmLT marker antigen did not interfere with the RESPISURE® efficacy or serological response for prevention of pneumonia from Mycoplasma hyopneumoniae and also had an additive adjuvant effect in that mean lung lesion scores were lower than those observed for RESPISURE® alone.

TABLE 4
Lung Lesions - 173 RCMV Study

| | LSM | SE | Rang |
|---|---|---|---|
| Placebo | 18.1 | 5.59 | 0.1-67 |
| rmLT old | 0.7 | 0.35 | 0-7.1 |
| rmLT new | 0.6 | 0.35 | 0-13.5 |
| RESPISURE ® | 1.1 | 0.47 | 0-4.95 |

What is claimed is:

1. A method of identifying an animal which has been vaccinated with an immunogen comprising:
a. combining said immunogen with a recombinant, substantially non-toxic E. coli heat-labile enterotoxin mutant (rmLT) to form a vaccine composition;
b. administering said vaccine composition to animal subjects; and
c. detecting the presence of antibodies or immune cells that are specific to said rmLT in said animal thereby identifying said animal as having been vaccinated with said vaccine preparation.

2. A method of determining that an animal that has been vaccinated with a vaccine composition which includes an immunogen and a substantially nontoxic rmLT, said method comprising detecting, in said animal, the presence of antibodies or immune cells that are specific to said rmLT.

3. A method of marking or identifying a vaccine composition containing an immunogen, comprising:
a. adding a substantially nontoxic rmLT in said vaccine composition;
b. administering said vaccine composition to an animal; and
c. detecting presence of antibodies or T cells that are specific to said rmLT in said animal thereby identifying said vaccine composition.

4. The method according to any one of claims 1-3, wherein said animal is a food animal selected from the group consisting of cattle, sheep, pig and poultry.

5. The method according to any one of claims 1-3, wherein said immunogen is a Mycoplasma hyopneumoniae immunogen.

6. The method according to claim 5, wherein said Mycoplasma hyopneumoniae immunogen comprises an inactivated, whole or partial Mycoplasma hyopneumoniae cell preparation.

7. The method according to claim 6, wherein said Mycoplasma hyopneumoniae cell preparation is RESPISURE®, RESPISURE ONE™, STELLAMUNE™ Mycoplasma or STELLAMUNE ONE™ Mycoplasma.

8. The method according to any one of claims 1-3, wherein said rmLT comprises the substitution of Gly for Arg at the amino acid position 192.

9. The method according to claim 8, wherein said rmLT further comprises the substitution of Ala for Leu at the amino acid position 211.

10. The method according to any one of claims 1-3, wherein said rmLT in said vaccine composition is in an amount of about 1-500 μg per dose.

11. The method according to claim 10, wherein said rmLT in said vaccine composition is in an amount of about 20-200 μg per dose.

12. The method according to claim 11, wherein said rmLT in said vaccine composition is in an amount of about 100 μg per dose.

13. The method according to claim 1 or 3, wherein the administration of said vaccine composition is via an oral, intranasal, mucosal, topical, transdermal, or parenteral route.

14. The method according to claim 13, wherein the administration of said vaccine composition is via an intramuscular route.

15. The method according to any one of claims 1-3, wherein said antibodies specific to said rmLT are detected in blood or milk of said animal or in meat juice from said animal after processing.

16. The method according to claim 15, wherein said antibodies are detected in an immunoassay.

17. The method according to claim 16, wherein said immunoassay is in the form of DIPSTICK, Western Immuno Blot or ELISA.

18. The method according to claim 16, wherein a peptide fragment of the A subunit of said rmLT is used in said assay.

19. The method according to claim 18, wherein said peptide fragment is selected from any one of SEQ ID NOS: 1-9.

20. A method for enhancing the immunoprotective effects of an immunogen in a vaccine composition for administration to an animal, comprising adding to said vaccine composition a substantially nontoxic rmLT.

21. The method according to claim 20, wherein an oil-in-water emulsion adjuvant is further added to the vaccine composition.

22. The method according to claim 21, wherein said oil-in-water emulsion adjuvant is AMPHIGEN®.

23. The method according to claim 20, wherein said animal is a food animal selected from the group consisting of cattle, sheep, pig, and poultry.

24. The method according to claim 20, wherein said immunogen is a Mycoplasma hyopneumoniae immunogen.

25. The method according to claim 24, wherein said Mycoplasma hyopneumoniae immunogen comprises an inactivated, whole or partial Mycoplasma hyopneumoniae cell preparation.

26. The method according to claim 25, wherein said Mycoplasma hyopneumoniae cell preparation is RESPISURE®, RESPISURE ONE™, STELLAMUNE™ Mycoplasma or STELLAMUNE ONE™ Mycoplasma.

27. The method according to claim 20, wherein said rmLT comprises the substitution of Gly for Arg at the amino acid position 192.

28. The method according to claim 27, wherein said rmLT further comprises the substitution of Ala for Leu at the amino acid position 211.

29. The method according to claim 20, wherein said rmLT in said vaccine composition is in an amount of about 1-500 μg per dose.

30. The method according to claim 29, wherein said rmLT in said vaccine composition is in an amount of about 20-200 μg per dose.

31. The method according to claim 30, wherein said rmLT in said vaccine composition is in an amount of about 100 μg per dose.

32. The method according to claim 20, wherein the administration of said vaccine composition is via an oral, intranasal, mucosal topical, transdermal, or parenteral route.

33. The method according to claim 32, wherein the administration of said vaccine composition is via an intramuscular route.

34. A vaccine composition for administration to an animal comprising an immunogen, a substantially non-toxic rmLT and an oil-in-water emulsion adjuvant.

35. The vaccine composition of claim 34, wherein said oil-in-water emulsion adjuvant is AMPHIGEN®.

36. The vaccine composition according to claim 34, wherein said animal is a food animal selected from the group consisting of cattle, sheep, pig, and poultry.

37. The vaccine composition according to claim 34, wherein said immunogen is a Mycoplasma hyopneumoniae immunogen.

38. The vaccine composition according to claim 37, wherein said Mycoplasma hyopneumoniae immunogen comprises an inactivated, whole or partial Mycoplasma hyopneumoniae cell preparation.

39. The vaccine composition according to claim 38, wherein said Mycoplasma hyopneumoniae cell preparation is RESPISURE®, RESPISURE ONE™, STELLAMUNE™ Mycoplasma or STELLAMUNE ONE™ Mycoplasma.

40. The vaccine composition according to claim 34, wherein said rmLT comprises the substitution of Gly for Arg at the amino acid position 192.

41. The vaccine composition according to claim 40, wherein said rmLT further comprises the substitution of Ala for Leu at the amino acid position 211.
